SEQUENCE LISTING (29 sequences)

SEQ ID NO: 1 - DNA, 22 bp, Artificial Sequence
  Description of Artificial Sequence  Synthetic Primer
tgcacttcgacgaccgcttctc

SEQ ID NO: 2 - DNA, 21 bp, Artificial Sequence
  Description of Artificial Sequence  Synthetic Primer
cgctgctcgagccgttttctc

SEQ ID NO: 3 - DNA, 20 bp, Artificial Sequence
  Description of Artificial Sequence  Synthetic Primer
ggtggctagctggaatcgcg

SEQ ID NO: 4 - DNA, 30 bp, Artificial Sequence
  Description of Artificial Sequence  Synthetic Primer
tgtacagctctcacacaaatttcattttgt

SEQ ID NO: 5 - DNA, 23 bp, Artificial Sequence
  Description of Artificial Sequence  Synthetic Primer
ggaattgctgactcagcattact

SEQ ID NO: 6 - DNA, 20 bp, Artificial Sequence
  Description of Artificial Sequence  Synthetic Primer
accacagtccatgccatcac

SEQ ID NO: 7 - DNA, 20 bp, Artificial Sequence
  Description of Artificial Sequence  Synthetic Primer
tccaccaccctgttgctgta

SEQ ID NO: 8 - DNA, 22 bp, Artificial Sequence
  Description of Artificial Sequence  Synthetic Primer
agcttccagaaggctgtcaagg

SEQ ID NO: 9 - DNA, 23 bp, Artificial Sequence
  Description of Artificial Sequence  Synthetic Primer
gctgccattcaatgcaatacgtc

SEQ ID NO: 10 - DNA, 24 bp, Artificial Sequence
  Description of Artificial Sequence  Synthetic Primer
agacacccacttatacactgagta

SEQ ID NO: 11 - DNA, 24 bp, Artificial Sequence
  Description of Artificial Sequence  Synthetic Primer
ctcttcttctggtgtttgtgctct

SEQ ID NO: 12 - DNA, 24 bp, Artificial Sequence
  Description of Artificial Sequence  Synthetic Primer
aaaagactctcttggtgccccttc

SEQ ID NO: 13 - DNA, 25 bp, Artificial Sequence
  Description of Artificial Sequence  Synthetic Primer
catagatctctccatcattgtccgc

SEQ ID NO: 14 - DNA, 20 bp, Artificial Sequence
  Description of Artificial Sequence  Synthetic Primer
accacagtccatgccatcac

SEQ ID NO: 15 - DNA, 20 bp, Artificial Sequence
  Description of Artificial Sequence  Synthetic Primer
tccaccaccctgttgctgta

SEQ ID NO: 16 - DNA, 30 bp, Artificial Sequence
  Description of Artificial Sequence  Synthetic Primer
ctcaagggcatcctgggctacactgagcac

SEQ ID NO: 17 - DNA, 31 bp, Artificial Sequence
  Description of Artificial Sequence  Synthetic Primer
tgtgggcttgctagttctagagccatgctcg

SEQ ID NO: 18 - DNA, 31 bp, Artificial Sequence
  Description of Artificial Sequence  Synthetic Primer
cacaagtcacacccagaaggttgatgcaggc

SEQ ID NO: 19 - DNA, 27 bp, Artificial Sequence
  Description of Artificial Sequence  Synthetic Primer
ctcccaatgcactgaaggcattccttg

SEQ ID NO: 20 - DNA, 29 bp, Artificial Sequence
  Description of Artificial Sequence  Synthetic Primer
gtccccttgcaaactctaccccccttaac

SEQ ID NO: 21 - DNA, 26 bp, Artificial Sequence
  Description of Artificial Sequence  Synthetic Primer
cgagaccgaataccaggtgatcggag

SEQ ID NO: 22 - DNA, 26 bp, Artificial Sequence
  Description of Artificial Sequence  Synthetic Primer
cttgtcctggaagaagcgcttgagcg

SEQ ID NO: 23 - DNA, 29 bp, Artificial Sequence
  Description of Artificial Sequence  Synthetic Primer
gacatcaaggagtccattgagaccatgcg

SEQ ID NO: 24 - DNA, 28 bp, Artificial Sequence
  Description of Artificial Sequence  Synthetic Primer
ctggaatctcagccccttctccaagacg

SEQ ID NO: 25 - DNA, 27 bp, Artificial Sequence
  Description of Artificial Sequence  Synthetic Primer
gttgccccacagttcacacactccctg

SEQ ID NO: 26 - DNA, 30 bp, Artificial Sequence
  Description of Artificial Sequence  Synthetic Primer
ggctcggcccggcaggacgagaaagagtgc

SEQ ID NO: 27 - DNA, 27 bp, Artificial Sequence
  Description of Artificial Sequence  Synthetic Primer
atggagtatgtcaacgacttcgacctg

SEQ ID NO: 28 - DNA, 25 bp, Artificial Sequence
  Description of Artificial Sequence  Synthetic Primer
acgactcacagaaagaactcgggag

SEQ ID NO: 29 - DNA, 29 bp, Artificial Sequence
  Description of Artificial Sequence  Synthetic Primer
ctgcggggcttcaccaaggacgaggtgat

(19) United States
(12) Patent Application Publication
(10) Pub. No.: US 2003/0138816 A1
(43) Pub. Date: Jul. 24, 2003

(54) Methods and kits for diagnosing the occurrence or the phase of minimal change nephrotic syndrome (MCNS) in a human

(75) Inventors: Djillali Sahali, Chatenay-Malabry (FR); Andre Pawlak, Saint Maur des Fosses (FR); Georges Guellaen, Villecresnes (FR); Philippe Lang, Paris (FR)

Correspondence Address: Mark B. Wilson, Fulbright & Jaworski L.L.P., Suite 2400, 600 Congress Avenue, Austin, TX, 78701, US

(73) Assignee: INSTITUT NATIONAL DE LA SANTE ET DE LA RECHERCHE MEDICALE, Paris (FR)

(21) Appl. No.: 10/266,008

(22) Filed: Oct. 7, 2002

Related U.S. Application Data
(60) Provisional application No. 60/327,603, filed on Oct. 5, 2001.

(51) Int. Cl. C12Q 1/68; G01N 33/53; G01N 33/567

(52) U.S. Cl. 435/6; 435/7.2

(57) ABSTRACT

Provided are methods and compositions for the diagnosis of the Minimal Change Nephrotic Syndrome (MCNS) in a human by detecting and/or quantifying a biochemical marker which is specific of the disease.

### CROSS-REFERENCE TO RELATED APPLICATIONS

This application claims priority to U.S. Provisional Application Serial No. 60/327,603 filed Oct. 5, 2001, the entire contents of which are specifically incorporated herein by reference.

### FIELD OF THE INVENTION

The present invention relates to the diagnosis of the Minimal Change Nephrotic Syndrome (MCNS) in a human by detecting and/or quantifying a biochemical marker which is specific of this disease.

### BACKGROUND OF THE INVENTION

Minimal Change Nephrotic Syndrome (MCNS) is a glomerular disease characterized by a heavy proteinuria with a relapsing/remitting course without histological evidence of classical immune mechanisms mediated injury. In these patients, persistent immunogenic stimuli such as viral infection, immunisation or allergen agents may trigger nephrotic relapses. During relapses, several immune cell subsets including CD4 and CD8 T cells are expanded, and cytokines such as TNFα, IL8, and IL13 are increased. Convincing evidence for an immune origin comes from sensitivity of MCNS to immunosuppressive therapy, but the molecular link between immune system and kidney disease is lacking. The inventors recently have shown that nuclear extracts of peripheral T lymphocytes from patients with relapse displayed a persistently high level of NF-KB DNA binding activity, which may account for the increased level of cytokines. In contrast, remissions are characterized by up regulation of IKB and down regulation of most of these cytokines.

Although the fundamental basis of T cell dysfunction remains unknown, it is believed that initiating mechanisms of the disease take place in the context of immune alterations affecting particular peripheral T cells, but nothing was known about these abnormalities.

The diagnosis of this disease is presently exclusively based on clinical observations. However, a diagnosis based on the sole detection of the clinical symptoms is difficult to perform and additionally is not specific, since the same clinical symptoms can be found in other nephropathies such as the membranous nephropathy (MN).

Additionally, the physiopathology of MCNS remains poorly understood, which explains why no biochemical marker of this disease was yet available in the art.

There is thus a need in the art for means allowing a quick and specific diagnosis of MCNS, for example a need for a specific biochemical marker of said disease and wherein said biochemical marker would be easily detectable.

### SUMMARY OF THE INVENTION

As it will be described in detail further in the specification, the inventors have now shown that the expression level of the c-Maf gene consists of the first specific biochemical marker of MCNS.

The present invention is primarily directed to the c-Maf gene expression detection or to the c-MAF protein detection/cell localization as biochemical markers for diagnosing the occurrence of MCNS in a human patient or for diagnosing MCNS remission phase versus MCNS relapse phase in a human patient who is affected with this disease.

The present invention is also directed to the use of means for detecting c-Maf gene expression and to the use of means for detecting the presence of the c-MAF protein in a biological sample for the purpose of diagnosing the occurrence of MCNS in a human patient or for diagnosing MCNS remission phase versus MCNS relapse phase in a human patient who is affected with this disease.

A first object of the invention consists of a method for diagnosing the occurrence of Minimal Change Nephrotic Syndrome (MCNS) in a human, wherein said method comprises the steps of:

a) collecting a biological sample from said patient;

b) quantifying the expression level of the c-Maf gene in the biological sample obtained at step a); and

c) comparing the expression level of the c-Maf gene quantified at step b) with the expected expression level of said gene in patients not affected with MCNS.

According to a first embodiment of the method described above, step b) consists of quantifying the mRNA transcribed from the c-Maf gene in said biological sample.

According to a second embodiment of the method described above, step b) consists of quantifying the c-MAF protein contained in said biological sample.

The present invention also relates to a method for distinguishing between a MCNS remission phase from a MCNS relapse phase in a human patient affected with Minimal Change Nephrotic Syndrome, wherein said method comprises the steps of:

a) collecting a biological sample from said patient; and

b) quantifying the c-MAF protein respectively in (i) the cell nucleus and (ii) in the whole-cell or the cell cytoplasm from the cells contained in said biological sample;

According to a first embodiment of the method described above, step b) of quantifying the c-MAF protein is performed by incubating at least one antibody which recognizes specifically the c-MAF protein with the cells contained in the biological sample and detecting the complexes formed between said antibody and the c-MAF proteins respectively localized within the nucleus and within the cytoplasm of said cells.

According to a second embodiment of the method described above, step b) of quantifying the c-MAF protein is performed by incubating at least one antibody which recognizes specifically the c-MAF protein with respectively (i) a nuclear extract and (ii) a whole cell extract or a cytoplasm extract obtained from the cells contained in the biological sample and detecting the complexes formed between said antibody and the c-MAF protein contained in said extracts.

According to a third embodiment of the method described above, step b) of quantifying the c-MAF protein is performed by incubating respectively (i) nuclear extracts and (ii) whole cell extracts or cytoplasm extracts obtained form the cells contained in the biological sample with a consensus Maf responsive element (MARE) probe and detecting the complexes formed between the MARE probe and the c-MAF proteins contained in said extracts.

The present invention is also directed to kits for performing the two general methods of diagnosis above.

### BRIEF DESCRIPTION OF THE FIGURES

FIG. 1: Strategy for the detection of genes differentially expressed in MCNS. Double stranded cDNA was synthesized from 1 μg of PBMC poly (A) RNA obtained from the same patient during the relapse and the remission phase. Subtractive hybridizations (forward and reverse), and PCR amplifications were performed as described under Material and Methods. Forward subtracted cDNA was cloned in pBluescript II SK (+) phagemid. Ten thousands clones of the library were screened using two types of probes: i) positive probes consisting of forward subtracted cDNA and unsubtracted relapse cDNA; ii) negative probes, including reverse subtracted cDNA, unsubtracted remission and MN cDNAs.

FIG. 2: Analysis of subtracted cDNAs. for β2-microglobulin sequence. Subtracted (a) and unsubtracted (b) cDNA were amplified by two rounds of PCR amplification. One-fifth of the first, and second PCR products were electrophoresed on a 1.5% agarose gel, transferred onto nylon filter and hybridized with a β2 microglobulin probe.

FIG. 3A, FIG. 3B and FIG. 3C. Differential screening of subtracted clones. Autoradiograms of identical filters containing sets of cDNA clones selected from cDNA library enriched in genes expressed in MCNS. The filters were hybridized with three [α³²P] dCTP labeled multiplex probes: FIG. 3A, forward subtracted probe, corresponding to cDNAs enriched in genes expressed in MCNS relapse; FIG. 3B, reverse subtracted probe, corresponding to cDNAs enriched in genes expressed in remission; FIG. 3C, first strand cDNA MN probe synthesized from 10 μg of PBMC total RNA. Arrowheads pinpoint clones expressed in MCNS (black) to a large extent than in reverse and MN (open).

FIG. 4A and FIG. 4B: FIG. 4A. Expression analysis in MCNS of subtracted transcripts corresponding to known genes. PBMC Total RNA pools from 4 relapses and 4 remissions, respectively, were spotted at 2 μg/slot onto a Hybond N membrane, and probed with subtracted cDNAs corresponding to L-Plastin, Grancalcin, TCR δchain, NFAT5, Proteasome α2 subunit (α2P). (Macropain) IL7 receptor, IgE dependent histamine releasing factor (HRF), and Jak1 transcripts. As control, one dot blot was hybridized with a glyceraldehyde phosphate dehydrogenase (GAPDH). FIG. 4B. Expression analysis in MCNS of subtracted transcripts corresponding to seven unknown clones. PBMC Total RNA pools from 7 relapses and 8 remissions, respectively, were spotted at 2 μg/slot onto a Hybond N membrane, and probed with cDNAs corresponding to clones SC1 to SC7.

FIG. 5A, FIG. 5B and FIG. 5C: Upregulation of the FYB mRNA expression in MCNS relapse. Relative expression of the FYB mRNA from: FIG. 5A. Patients with MCNS (n=7) during the relapse off steroids and in remission phases; FIG. 5B. Patients with membranous nephropathy (n=5); FIG. 5C. Controls (n=5). Semiquantitative reverse transcription-PCR was performed with 2 μg of total RNA as described under material and methods. The expression of GAPDH was monitored in parallel, in order to control the variation in RT reaction.

FIG. 6A, FIG. 6B and FIG. 6C: Downregulation of the IL12Rβ2 subunit mRNA expression in MCNS relapse. Relative expression of the IL12Rβ1 and IL12Rβ2 subunits mRNA from: FIG. 6A. Patients with MCNS (n=7) during the relapse off steroids and in remission phases; FIG. 6B. Patients with membranous nephropathy (n=5); FIG. 6C. Controls (n=5). Semiquantitative reverse transcription-PCR was performed with 2 μg of total RNA as described under material and methods. The expression of GAPDH was monitored in parallel, in order to control the variation in RT reaction.

FIG. 7: Specific induction of short (Maf-S) and long (Maf-L) cmaf mRNA during the relapse MCNS. In most patients, the induction of cmaf primarily involves the short form. Reverse transcription was performed with 2 μg of total RNA as described under material and methods, from nine patients studied in relapse and in remission. Semiquantitative-PCR was performed with fifty ng of cDNA, using primers listed in table 1. After Southern blotting, the PCR products were detected with specific probes. The expression of GAPDH was monitored in parallel, in order to control the variation in RT reaction.

FIG. 8: Induction of cmaf was higher in the CD4 T cell subset. T cell subsets were purified from PBMC as described under material and methods. Total RNA was prepared and analyzed for cmaf expression by semiquantitative reverse transcription-PCR in four patients. The expression of GAPDH was monitored in parallel, in order to control the variation in RT reaction.

FIG. 9A, FIG. 9B and FIG. 9C: FIG. 9A. Induction of cmaf in MCNS relapse was not associated with an up regulation of the transcription factor GATA3. Reverse transcription was performed with 2 μg of total RNA as described under material and methods, from nine patients studied in relapse and in remission. Semiquantitative-PCR was performed with fifty ng of cDNA, using primers listed in table 3. After Southern blotting, the PCR products were detected with specific probes. The expression of GAPDH was monitored in parallel, in order to control the variation in RT reaction. FIG. 9B. Quantification of PCR products as determined by the Image Quant V 1.11 analysis software, after normalization against the corresponding GAPDH mRNA values. FIG. 9C. Statistical analysis of densitometric values, using the ANOVA program (P>0,05)

FIG. 10: Lack of induction of the transcription factor MafB in most patients with MCNS relapse. Reverse transcription was performed with 2 μg of total RNA as described under material and methods, from seven patients studied in relapse and in remission. Semiquantitative-PCR was performed with fifty ng of cDNA, using primers listed in table 3. After Southern blotting, the PCR products were detected with specific probes. The expression of GAPDH was monitored in parallel, in order to control the variation in RT reaction.

FIG. 11A and FIG. 11B: FIG. 11A. Nuclear translocation of cmaf protein during MCNS relapse. Immunodetection of nuclear cmaf proteins in relapse and remission of MCNS in four patients. 10-20 μg of nuclear extracts were analyzed by Western blot using antibodies raised against cmaf. FIG. 11B. Immunodetection of cytosolic cmaf proteins in relapse and remission in seven patients with MCNS, as well as in five normal subjects. Proteins (50 μg) from cytosolic extracts were analyzed by Western blot using antibodies raised against cmaf. The positions of cmaf-S (lower band of doublet) and cmaf-L (upper band of the doublet) are indicated. The blots were subsequently stripped and reprobed with anti-actin antibodies.

FIG. 12A, FIG. 12B and FIG. 12C: DNA binding activity of cmaf protein in MCNS relapse. Nuclear extracts were prepared from PBMCs as described under materials and methods. 10-20 μg of nuclear proteins from six patients in nephrotic relapse (FIG. 12A) and then in remission (FIG. 12B), were incubated with the wild type MARE oligonucleotide. The specificity of the band shift was demonstrated by the loss of this band, in the presence of 50 fold excess of unlabelled MARE oligonucleotide. FIG. 12C. Identification of DNA-protein complexes. 20 μg of nuclear extracts isolated from PBMC of one patient with MCNS relapse were analyzed in EMSA for MARE site in the absence (−) or presence (+) of antibodies indicated. Supershifted bands (indicated by arrow) were present when anti-cmaf, anti-cfos or anti-cjun antibody was incubated with extracts before probe addition.

FIG. 13: Immunocytochemical localization of c-maf during MCNS disease. The upper panels represent the expression of c-maf in either PBMC or CD4 T cells of patients and normal controls. The lower panels identify the location of the cell using DAPI staining.

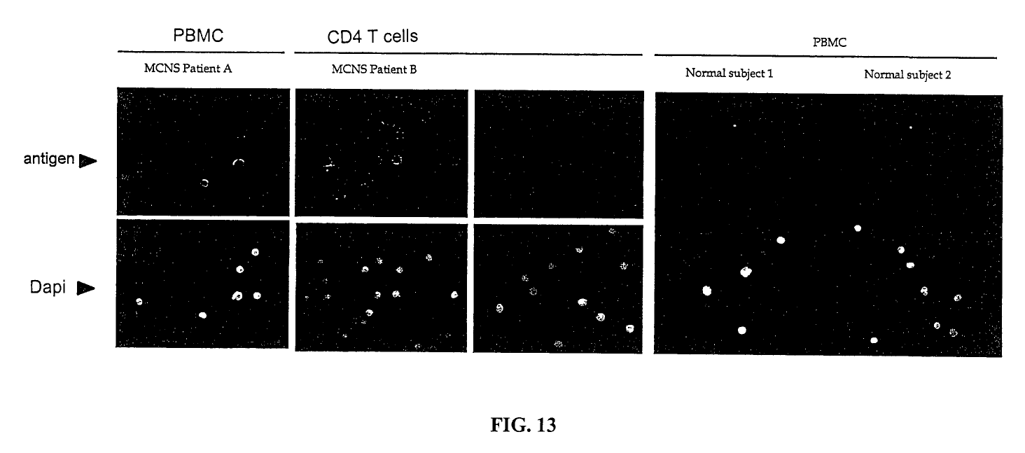

### DETAILED DESCRIPTION OF THE INVENTION

In order to identify the molecular mechanisms occurring in peripheral T cells during MCNS relapse, the inventors have undertaken a subtractive and differential cloning of transcripts selectively induced or upregulated in lymphocytes of patients with MCNS relapse. To this end, the inventors purified T cell-enriched PBMC from a patient at the time of relapse and of remission, and established a subtractive cDNA library. Differential screening of this library led to the identification of 84 clones. At least 18 clones encode parts of genes involved in tightly coordinate steps of T cell activation, supporting the hypothesis that MCNS is a T cell-mediated disease. Furthermore, the inventors showed that this T cell response is associated with a downregulation of IL12Rβ2 mRNA levels, suggesting that a Th2 phenotype occurs early in the course of this disease.

By partial sequencing of subtracted CDNA clones, the inventors have identified the proto-oncogene c-maf, a member of the basic region/leucine zipper transcription factor family. Based on structural and functional properties, Mafs may be subdivided in two subfamilies. Big mafs include the c-Maf proto-oncogene, neural retinal specific gene (NRL), and Maf B. They are characterized by an N amino-terminal proline/serine/threonine-rich acidic transactivation domain and a carboxy-terminal basic region containing the DNA binding domain. Although big mafs family members exhibited similar DNA binding, they are different functions and the target genes are distincts. This specificity is likely retained within the region proximal to DNA binding domain. C-Maf binds to a palindromic sequence named Maf recognition element (MARE) that consist of an extended AP1 motif but the consensus binding sequence is not well established. C-maf binds to target sequences as homo or heterodimere with fos or jun but do not dimerize with small mafs. In contrast to large mafs, small mafs, including maf F, maf K, and mat G, consist primarily of basic region and lack the N terminal transactivation domain. This last characteristic, as well as experimental evidence based on reporter assays, suggest that small mafs, when they dimerize with themselves, may act as natural repressors of large maf-induced transcription. Currently, few cellular genes are known to be regulated by c-maf. In response to TCR activation, polarization of precursor T cells toward Th2 subset is associated with a selective induction of c-maf which, in return, transactivates the IL 4 gene. Although evidence supporting the role of cmaf in IL4 production has been documented, it is become clear that the IL4 induction may be independent of cmaf. For example, retroviral expression studies have shown that Gata-3-, but not c-maf- or Jun-B-expressing virus, induces IL4 production and inhibits IFN-γ(KM), and c-maf does not increase the endogenous expression of Gata-3.

Two forms of c-maf mRNA have been isolated (blood). The 4248 bp mRNA especes encodes a predicted short protein of 373 aa (S/c-maf). The 2145 bp mRNA especes is generated by alternative splicing which leads to a losing the 3′UTR and inserting an unique exon located at 2,1 kb downstream of the polyadenylation signal. It encodes a predicted long form protein of 403 aa (L/c-maf) and is identical to v-maf, excepted a point mutation at position x. Whether L-and short forms of c-maf exhibited similar or distinct functional roles is not known. Due to very modest expression of c-maf in normal subjects and the lack of knowledge about the downstream target genes, few data are available concerning the regulation of c-maf in normal and pathological conditions. The isolation of c-Maf by subtractive cloning allowed us to investigate its relevance in MCNS.

The inventors report for the first time that the c-Maf was specifically induced in MCNS relapse, but not detected in normal subjects or membranous nephropathy patients. The induction of c-Maf both at mRNA and protein levels, was selectively restricted to short form (S/c-maf). The inventors showed that the S/c-maf protein was detected in nucleic fractions during the relapse phase but only in cytosol during the remission. In contrast, the L/c-maf protein displayed no significant changes during relapse and remission phases. The inventors also demonstrated that the co culture of PBMC from relapse with the proteasome inhibitor MG132 induces a sequestration of S/c-maf protein whereas no evident change of L/c-maf protein was detected. These results suggest that in patients with nephrotic syndrome, a strong induction of cmaf in peripheral lymphocytes is compatible with the diagnostic of MCNS. The induction of c-Maf was not correlated with an induction of IL4 gene, suggesting that the downstream target gene remains to be identified.

Thus, a first object of the invention is to provide with methods and kits allowing the diagnosis of the occurrence of the MCNS in a human patient.

The present invention is directed to a method for diagnosing the occurrence of Minimal Change Nephrotic Syndrome (MCNS) in a human, wherein said method comprises the steps of:

a) collecting a biological sample form said patient;

b) quantifying the expression level of the c-Maf gene in the biological sample obtained at step a); and

c) comparing the expression level of the c-Maf gene quantified at step b) with the expected expression level of said gene in patients not affected with MCNS.

According to a first embodiment of the method above, step b) consists of quantifying the mRNA transcribed from the c-Maf gene in said biological sample.

Since the nucleotide sequence is part of the prior art, the one skilled in the art is able to quantify easily the mRNA transcribed from the c-Maf gene, according to his general technical knowledge of the numerous techniques described in the art.

For example, the one skilled in the art can quantify the mRNA transcribed from the c-Maf gene by RT-PCR as it is disclosed in the examples.

In a first aspect of the first embodiment of the method above, the step of quantifying the mRNA transcribed from the c-Maf gene is performed by a RT-PCR reaction using a pair of primers hybridizing specifically with the c-Maf cDNA.

Most preferably, the primers used for RT-PCR are the primers consisting of the nucleic acid of sequences SEQ ID NO:1 to SEQ ID NO:4.

Preferably, the biological sample is a body fluid containing cells of the patient to be tested.

More preferably, the biological sample is a body fluid containing mononuclear cells from the immune system of said patient, advantageously T and B cells, and better T cells.

Most preferably, the biological sample consists in a whole blood sample or a sample containing mostly peripheral blood mononuclear cells (PBMC) which can be obtained after cell isolation or purification from a whole blood sample.

In a second embodiment of the method above, step b) consists of quantifying the c-MAF protein contained in said biological sample.

According to a first aspect of said second embodiment, the step of quantifying the c-MAF protein is performed by incubating at least one antibody which recognizes specifically said protein with said biological sample.

This can be performed through any procedure allowing the detection of antigen-antibody complexes well known from the one skilled in the art, such as ELISA (Enzyme-linked immunosorbent assay), RIA (Radio-immunoassay) or FIA (Fluorescence immunoassay).

Advantageously, the detection of the specific antigen-antibody complexes is performed through an immunofluorescence assay by using an antibody recognizing specifically the c-MAF protein and wherein said antibody is fluorescently labeled, either directly or indirectly. The antibody is directly labeled when it is conjugated to a fluorescent detectable molecule. The antibody is indirectly labeled when the fluorescent molecule is added onto said antibody after the achievement of the antigen-antibody complexes. Such indirectly labeled antibodies encompass biotin-conjugated antibodies onto which a conjugate of streptavidin-fluorescent molecule may be added to fluorescently label it.

According to another aspect of said second embodiment, the step of quantifying the c-MAF protein is performed by incubating at least one antibody which recognizes specifically said protein with a protein extract obtained from said biological sample and detecting the complexes formed between said antibody and the c-MAF protein contained in the biological sample.

The protein extract may be obtained from the cells which are contained in the biological sample collected from the human patient tested, according to procedures which are well known from the one skilled in the art, for example the procedures disclosed in the examples.

According to a further aspect of said second embodiment, the step of quantifying the c-MAF protein is performed by incubating at least one antibody which recognizes specifically said protein with cells which are contained in said biological sample and detecting the localization of the complexes formed between said antibody and the c-MAF protein within said cells. This further aspect encompasses the immuno-histochemistry procedures well known from the one skilled in the art, such as those described in the examples.

Preferably, the cells contained in the biological sample collected from the tested patient consist of PBMCs.

According to a third embodiment of the diagnosis method described above, the step of quantifying the c-MAF protein is performed by incubating said biological sample consisting of a protein extract with a consensus Maf responsive element (MARE) probe and detecting the complexes formed between the MARE probe and the c-MAF proteins contained in the biological sample.

Most preferably, the MARE probe consists of the nucleotide sequence SEQ ID NO:5.

In another aspect of the invention, the inventors have also shown that the localization of the c-MAF protein within the cells was differentially regulated. More specifically, the inventors have shown that the short form of the c-MAF protein was detected in the cell nucleus in the biological samples collected from patients undergoing a relapse phase of the disease, whereas the short form of the c-MAF protein was detected exclusively in the cell cytosol during a remission phase.

The present invention is thus further directed to a method of in vitro diagnosis for distinguishing between a MCNS remission phase from a MCNS relapse phase in a human patient affected with Minimal Change Nephrotic Syndrome which includes a step of detecting and selectively localizing the short form of the c-MAF protein contained within the cells comprised in the biological sample of the patient under testing.

This invention also relates to the use of the short form of the c-MAF protein as a biochemical marker for determining the phase of the disease, namely a remission phase or a relapse phase.

Thus, another object of the invention consists of a method for distinguishing between a MCNS remission phase from a MCNS relapse phase in a human patient affected with Minimal Change Nephrotic Syndrome, wherein said method comprises the steps of:

a) collecting a biological sample from said patient; and

b) quantifying the short form of the c-MAF protein respectively in (i) the cell nucleus and (ii) in the whole-cell or the cell cytoplasm from the cells contained in said biological sample.

According to a first embodiment of the method above, step b) of quantifying the short form of the c-MAF protein is performed by incubating at least one antibody which recognizes specifically the c-MAF protein with the cells contained in the biological sample and detecting the complexes formed between said antibody and the short form of c-MAF protein respectively localized within the nucleus and within the cytoplasm of said cells.

Preferably, the cells consist of PBMCs.

According to a second embodiment of the method above, step b) of quantifying the short form of the c-MAF protein is performed by incubating at least one antibody which recognizes specifically the the c-MAF protein with respectively (i) a nuclear extract and (ii) a whole cell extract or a cytoplasm extract obtained from the cells contained in the biological sample and detecting the complexes formed between said antibody and the short form of the c-MAF protein contained in said extracts.

Most preferably the detection of the short form of the c-MAF protein is performed through a Western blot technique wherein the bands corresponding respectively to the short and long form of the c-MAF protein are easily visualized, as it is disclosed in the examples.

Preferably, the cells consist of PBMCs.

According to a third embodiment of the method above, step b) of quantifying the c-MAF protein is performed by incubating respectively (i) nuclear extracts and (ii) whole cell extracts or cytoplasm extracts obtained form the cells contained in the biological sample with a consensus Maf responsive element (MARE) probe and detecting the complexes formed between the MARE probe and the c-MAF proteins contained in said extracts.

Preferably, the MARE probe consists of the nucleotide sequence SEQ ID NO:5.

The present invention also relates to a kit for diagnosing the occurrence of Minimal Change Nephrotic Syndrome (MCNS) or for diagnosing the occurrence of a MCNS remission phase or a MCNS relapse phase in a human, wherein said kit comprises an antibody which recognizes specifically the c-MAF protein.

Preferably, said antibody is labeled with a detectable molecule.

Most preferably, said antibody is labeled with a fluorescent molecule.

The present invention is also directed to a kit for diagnosing the occurrence of Minimal Change Nephrotic Syndrome (MCNS) or for diagnosing the occurrence of a MCNS remission phase or a MCNS relapse phase in a human, wherein said kit comprises a consensus Maf responsive element (MARE) probe.

Preferably, the MARE probe consists of the nucleotide sequence SEQ ID NO:5.

The invention also concerns a kit for diagnosing the occurrence of Minimal Change Nephrotic Syndrome (MCNS) in a human, wherein said kit comprises a pair of primers hybridizing specifically with the c-Maf cDNA.

The present invention is further illustrated, without in any way being limited to, the examples below.

### EXAMPLES

Material and Methods for Examples 1 and 2

1) Patients: The cohort of patients analyzed in this study has been already described (5). In children, the criteria of the international study of kidney disease were used for diagnostic and management of MCNS (6). In adults, the diagnostic of MCNS or Membranous Nephropathy (MN) was confirmed by renal biopsy before inclusion. Blood sampling of patients with relapse was performed before any treatment except when indicated. Informed consent was obtained from the parents and whenever possible from the pediatric patients, as well as from normal volunteers.

The patient selected for construction of the subtractive library was a 24-year-old Caucasian man who was referred to us at the onset of nephrotic syndrome. Clinical examination showed oedema, but no other anomalies were noted. Blood pressure was 130/70. Laboratory assessment showed the following: serum creatinine, 63 μm/l; hemoglobin, 1.4 g/di; white blood cell count, 5,500/ml; platelets, 250,000/ml; serum albumin, 2.3 g/dl; proteinuria was 30 g/day, of selective type; urinary white and red blood cells less than 5000/ml, leucocyturia was negative. Serum complement was normal, serum IgG level was low (4 dg/dl), whereas the serum IgE level was increased (400 KUI). Tests for serum anti nuclear antibodies, anti streptolysin O, anti-double-stranded DNA, anti-neutrophil cytoplasm antibodies and cryoglobulinemia, were negative. Serologies were negative for EBV, yersinia, Lyme disease, syphilis, B and C hepatitis, and positive for CMV (of IgG type). Renal histological examination showed minimal changes on 25 glomeruli, without mesangial cell proliferation, interstitial cell infiltration, or focal glomerulohyalinosis. Immunofluorescence staining for immunoglobulin IgA, IgG, IgM, Complement 3 and 4, and fibrinogen were negative.

The patient was treated with prednisone (1 mg/kg/day) for four weeks and dosage was then progressively tapered. Complete remission was obtained within ten days after the beginning of treatment.

Blood samples from this patient were obtained in relapse, before initiation of steroid therapy, and two months after remission, while receiving 20 mg/day of prednisone therapy.

2) Construction of forward (“Relapse minus Remission”) and reverse (“Remission minus Relapse”) subtracted cDNAs: Peripheral blood samples were taken in relapse and in remission phases and the mononuclear cell fraction (PBMC) was isolated through a Ficoll/Hypaque gradient. T cell enriched population was obtained by filtering the PBMC suspension through a nylon wool column. Total RNA was prepared using the Qiagen kit (Qiagen SA), according to the manufacturer's instructions. The integrity of RNA was checked by running a 1 μg sample on 0.8% agarose gel followed by ethidium bromide staining. Poly (A) RNAs were purified using the oligotex mRNA kit (Qiagen). “Relapse” and “Remission” cDNA syntheses were performed in parallel with 1 μg of poly (A) RNA each, using the reverse transcriptase SII (Gibco BRL) and primers provided in the PCR-select cDNA subtraction kit (Clontech). Both cDNA populations were digested with the restriction enzyme Rsal to obtain shorter, blunt end molecules (7).

The forward subtracted cDNA was prepared as follows: the “Relapse” cDNA products were subdivided into two parts and each was ligated to a different adaptor. Fractions of each adaptor-linked “Relapse” cDNA were mixed and used as unsubtracted cDNA control. Subtractive hybridization was performed in two successive rounds, as follows: first, each adaptor-linked cDNA population was separately mixed with a 60-fold excess of “remission” cDNA. After denaturation for 90 sec at 98° C., subtractive hybridization was performed for 8 hours at 68° C. This first hybridization round equalizes the levels of different transcripts and enriches for sequences specifically expressed in relapse. Then, both reaction products were mixed and a five-fold excess of denatured “remission” cDNA was added. A second hybridization was performed for 16 hours at 68° C. During this step, single strand cDNAs, specific of the relapse phase and bearing different adaptors, formed hybrids which were subsequently amplified by two rounds of PCR. In the first PCR (24 cycles), only hybrid cDNAs were exponentially amplified. The second-nested PCR (12 cycles) enriched these specific sequences while reducing the background.

The reverse subtracted cDNA was prepared using the same protocol but switching the relapse and remission cDNA. To check the efficiency of the subtraction, an aliquot of both subtracted and unsubtracted cDNAs was blotted and probed with a 550-bp PstI β2-microglobulin fragment, following standard protocols (8).

3) Cloning of the forward subtracted cDNA into pbluescript II SK (+) phagemid vector: The amplified forward subtracted cDNA was blunt-ended, ligated to XhoI-NotI adaptors (Stratagene), and inserted into the NotI digested pBluescript II SK (+) phagemid (Stratagene) (8). Supercompetent E. coli XL-1 blue cells (Stratagene) were transformed with an aliquot of the ligation mix by heat shock (8).

One fraction of the library (10,000 colonies) was spread on nitrocellulose filters. Each master filter was duplicated twice, and stored at −20° C. while the duplicates were treated for differential screening (8).

Preparation of probes and differential screening: Two μg of subtracted cDNA (forward and reverse) were sequentially digested with RsaI, SmaI and EagI restriction enzymes, in order to remove all adaptors. After purification on a chromaspin-100 (Clontech), 80 ng of the digested cDNA were labeled with 5 μCi of [³²P] dCTP (3000 Ci/mmol, Amersham). Multiplex probes from unsubtracted, relapse and remission MCNS, as well as MN, were prepared from 10 μg PBMC total RNA as described (9).

Each duplicate filter of the subtracted cDNA library was incubated with the forward or reverse subtracted probe (2.10⁶ cpm/ml) respectively, at 72° C. for 16 h, washed 4×30 min in 2×SSC (SSC: 150 mM Nacl, 15 mM sodium citrate, pH 7), 0.5% SDS and 3×30 min in 0.2×SSC, 0.5% SDS at 68° C. After exposure, filters were dehybridized by boiling in 0.5% SDS for 10 min, and then rehybridized with unsubtracted multiplex probes as above.

4) RNA dot-blot analysis: PBMC total RNAs, isolated from patients in relapse off steroids, and in remission, were pooled separately and denatured at 65° C. Then, 2 μg of each mix were dot-blotted on Hybond-N membrane (Amersham). The membranes were washed twice in 6×SSC and cross-linked by UV exposure. Hybridization was performed in 5×SSPE (SSPE: 150 mM Nacl, 10 M NaH₂PO₄H₂O, 1 mM EDTA), 50% deionized formamide, 2× Denhardt's reagent (1× Denhardt's reagent: 0.1% Ficoll type 400 (Pharmacia), 0.1% Polyvinylpyrrolidone, 0.1% BSA), 0.5% SDS, salmon sperm DNA (100 μg/ml), and 2.106 cpm/ml of selected clones. In parallel, the sensitivity of hybridization was checked by using a glyceraldehyde phosphate dehydrogenase (GAPDH) probe.

5) DNA sequencing. Preparation and sequencing of double stranded plasmid DNA template and sequencing were performed as previously described (10). Nucleic acid and protein database searches were performed using resources of the National Center Biotechnology Information.

6) Semiquantitative reverse transcription PCR (RT-PCR). The sequence of primers, and main characteristics of PCR are indicated in table 1. The expression level of FYB, IL12Rβ1 and IL12Rβ2 subunits were analyzed by semiquantitative RT-PCR as previously described (5). Southern blots of amplified products were detected with specific internal oligonucleotides. PCR reactions were normalized for GAPDH expression, in order to control the variations in the RT reaction.

### Example 1

### Construction and Differential Screening of the Forward Subtracted cDNA Library

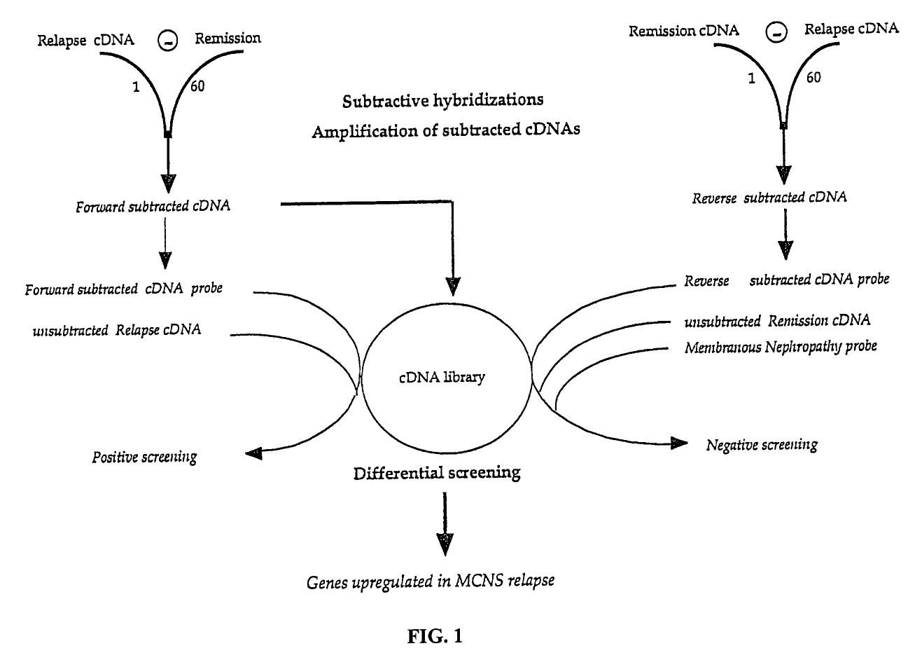

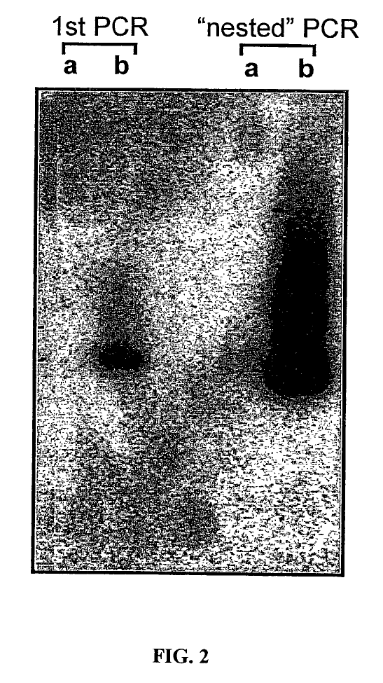

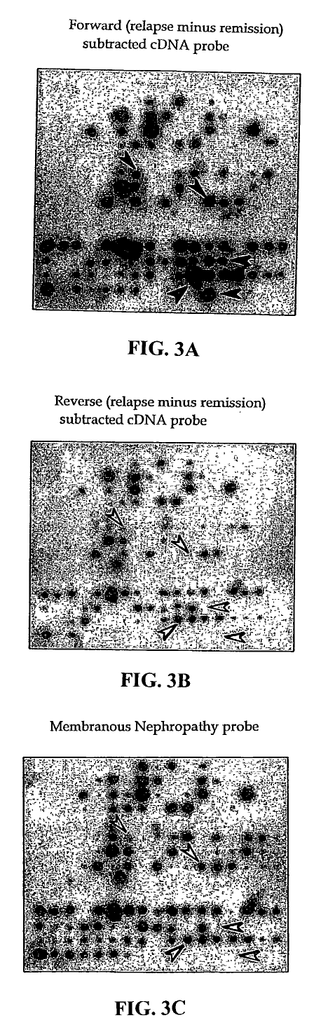

T cell dysfunction in MCNS involves the turning on and off of a number of genes likely to play a key role in the development of the disease. To identify these genes, the inventors isolated cDNAs corresponding to mRNAs of which, the expression was newly induced, and/or upregulated in T cells from a MCNS patient with nephrotic relapse. To achieve this goal, the inventors followed the strategy outlined on FIG. 1. As a first step, the inventors performed a subtractive hybridization between cDNAs of T cell-enriched PBMC from relapse versus remission. This was done from the same patient in order to obviate differences among individuals. The forward subtracted cDNA population, enriched in relapse-induced cDNAs, was cloned in pBluescript II SK (+) phagemid and 10,000 clones were analyzed by differential hybridization with the following probes: i) for a positive screening: forward subtracted and first strand “relapse” cDNA probes; ii) for a negative screening: reverse subtracted cDNA, first strand “remission” cDNA and MN probes, respectively. The efficiency of the subtraction was assessed by analyzing the β2-microglobulin expression in subtracted and unsubtracted samples. As shown in FIG. 2, the β2-microglobulin probe gave a strong signal with the unsubtracted double-stranded cDNAs, but no signal was detectable in the forward subtracted templates (FIG. 2). As expected, hybridization of the library to the forward subtracted probe revealed most of the clones, with a variable intensity (FIG. 3A). In contrast, 40% of the clones did not hybridize to the reverse subtracted probe and 45% of the other clones corresponded to downregulated mRNAs (FIG. 3B). Hybridization of the library with the MN probe showed that 75% of clones exhibited a significant signal, indicating that these clones corresponded to genes likely to be upregulated in response to the nephrotic state, independently of MCNS (FIG. 3C). Finally, 127 clones appeared selectively upregulated and/or exclusively expressed during MCNS relapse and were considered as relevant to the disease and retained for further analysis.

### Example 2

### DNA Sequence and Expression Analysis of the Subtracted Clones

A partial sequence of these 127 cDNAs was determined and compared to sequences available in databases (Table 2). Sequences obtained from 97 clones corresponded to 54 known human genes. The very low redundancy of these sequences, including sequences related to abundant transcripts such as β actin or Elf 1α underlined the efficiency of the subtraction strategy. Among these transcripts, 12 had no database match with proteins of assigned function. Thirty sequences did not exhibit any match with sequences in databases, they correspond to so far unidentified genes.

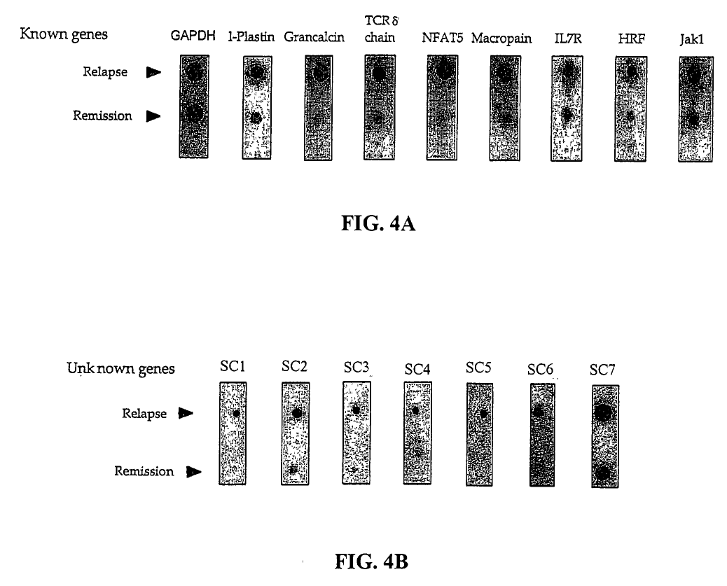

In order to confirm that these transcripts actually represent genes upregulated in MCNS relapse, the inventors analyzed eight, corresponding to known genes involved in TCR signaling pathway. In parallel, the inventors analyzed the expression of seven transcripts with no matches in databases. Dot blots loaded with pools of PBMC total RNA from patients with nephrotic relapse off steroids and patients in remission were hybridized with radiolabelled cDNA inserts corresponding to selected transcripts. As shown in FIG. 4A, the mRNA level of L-Plastin, Grancalcin, TCR δ chain, NFAT5, Macropain, IL7 receptor, IgE dependent histamine releasing factor (HRF), and Jak1 was increased in relapse and low or undetectable in remission. In comparison, a similar signal intensity was obtained with the GAPDH probe.

Transcripts corresponding to seven unknown genes displayed a high expression in PBMC from patients with nephrotic relapse (FIG. 4B). For three clones (SC1, SC4, SC5), no signal was detected in samples from remission. For the SC7 clone, a significant signal was detected in remission, although to a lesser extent than in relapse. The high dot blot signal obtained in relapse reveals that the selected sequences corresponded to transcripts highly induced during active MCNS. The complete sequencing of these clones is now in progress. The unknown genes found among the selected clones might encode new proteins susceptible of playing a key role in MCNS. Altogether, these results indicate that the forward subtracted cDNA library is highly enriched in sequences specifically upregulated in MCNS.

At least 18 genes identified in this library are involved in the TCR-mediated complex signaling cascade. The initial event of TCR activation consists in the ligation of T cell receptor with its cognate peptide ligand coupled to class I MHC antigen. T cell activation induces cytoskeleton reshaping that stabilize interactions between T cell and antigen presenting cells, and which function as a driving belt of TCR-mediated signals (11). L-plastin and grancalcin, identified in this work, are among proteins involved in cytoskeleton rearrangement. L-plastin, recruited following engagement of the TCR/MHC complex, mediates co stimulatory signals through associated receptors such as CD2 and is functionally associated with cytokine secretion (12). Grancalcin is a calcium binding protein, belonging to the family of EF hand proteins. Recently, interaction of grancalcin and L-Plastin has been documented (13). Along the same line, the inventors also identified Rho A that is a member of the Rho family that control cytoskeleton changes by acting on their protein kinase downstream targets (14).

Two transcripts IL7Rα and Jak1, displaying higher expression in MCNS relapse, are physically associated and cooperate to mediate signaling in response to IL7 and thymic stromal lymphopoetin, two cytokines involved in B lymphopoiesis and T cell development. The IL7R signaling pathway depends on Jak1, as supported by the lack of IL7-induced proliferation in thymocytes from Jak1 deficient mice (15). Both IL7R and Jak1 are recruited in multiple signaling pathways involving different cytokines or growth factors (16).

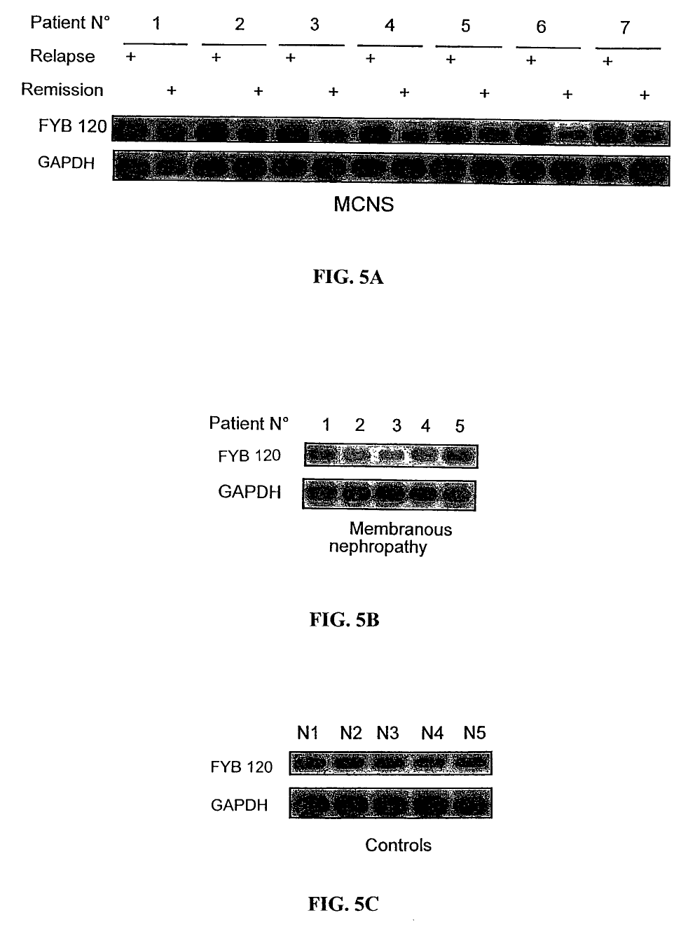

Following engagement of the TCR complex, activation of downstream signal pathways requires tyrosine phosphorylation of TCR by proximal kinases, p₅₆^{lck} and ZAP 70, which bridge the receptor complex with downstream adaptor proteins such as Fyb 120, a Fyn-T binding protein that was identified in the inventors' subtracted library. FYB binds to the SH2 domain of the Src kinase Fyn-T and SLP-76 (17). The expression of FYB is restricted to cells of hematopoietic lineages, including T cells but not B cells (17). The inventors analyzed the expression of FYB mRNA during the relapse and remission phases by semiquantitative RT-PCR in seven patients. As depicted in FIG. 5A, FIG. 5B and FIG. 5C, FYB mRNA levels were significantly increased in relapse as compared to remission and controls. This result cannot be explained by immunological perturbations induced by the nephrotic state itself since in MN-linked nephrotic syndrome, the expression of FYB was similar to normal controls. The inventors suggest that the recruitment of FYB may represent a key step in the propagation of specific TCR signaling in MCNS.

Adaptor proteins connect the TCR signal with gene transcriptional pathways. Indeed, the activation of target genes by transcription factors represents the effector arm of TCR signaling. According to the type of immune challenge, specific transcriptional factors are recruited, inducing particular immune responses. Along the same line, several transcription factors, such as NFAT5, c-maf, AP2 beta, NFRKB, were identified in this work. The target genes of most of these factors are unknown. The inventors' results suggest that TCR-induced T cell activation occurs in MCNS relapse. The increased expression of NFAT5 in this context is not surprising (18). As for many transcription factors, the upregulation of NFAT5 mRNA is associated with the induction of its protein product, otherwise undetectable in resting T cells (18).

Several subtracted transcripts, including IL1 beta, Rantes, p38 MapK have been implicated in regulating signals, leading to the activation of NFKB. The increase of proteasome α2 subunit mRNA levels is correlated with the increase in proteasome activity that contribute for a sustained activity of NF-KB in MCNS relapse (5).

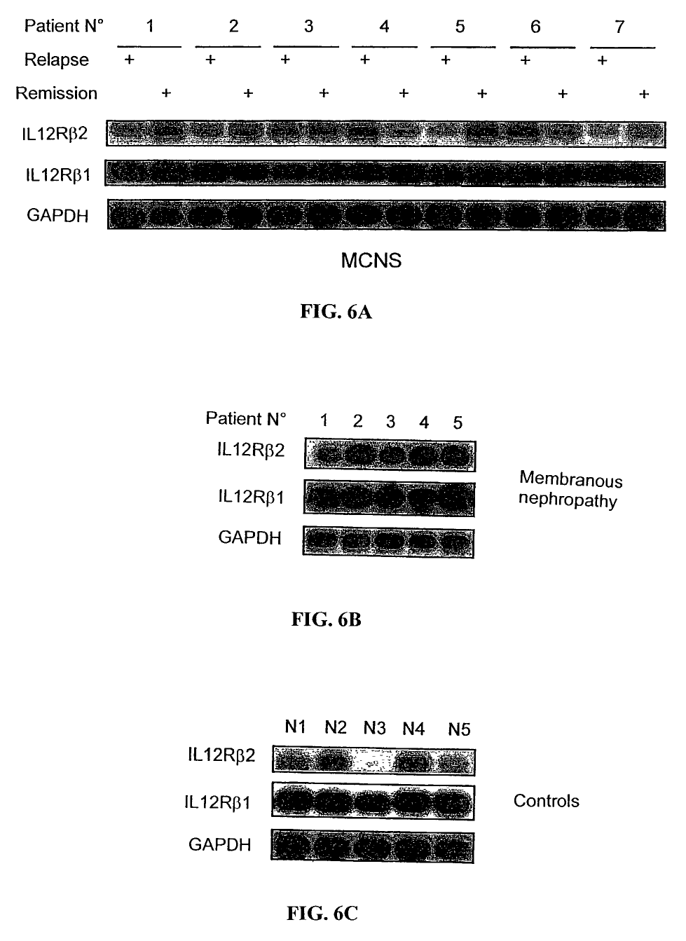

The inventors sought to determine whether T cell activation in MCNS relapse was associated with a particular Th phenotype. The IL12R complex, which consists of β1 and β2 subunits, mediates the IL12 signaling pathway of which the turn off occurs during differentiation of naive T cells into Th2 but not Th1 populations (19, 20). Humans Th1 cells express selectively the IL12Rβ2 transcript that is the signaling component of 1112R. The expression of IL12R transcripts was serially analyzed in seven children patients with MCNS, whose PBMC samples, obtained without exogenous stimulation, were available in relapse off steroids and in remission phases. As shown in FIG. 6A, FIG. 6B and FIG. 6C, levels of IL12R β1 transcripts were similar in MCNS patients, both in relapse and in remission, as well as in MN patients and normal controls. By contrast, the IL12Rβ2 transcripts were expressed at a very low level in relapse. The MN patients exhibited higher levels of the IL12Rβ2 transcripts compared to normal controls. Therefore, the downregulation of IL12 signaling pathway suggests that the MCNS is a Th2-mediated disease. Interestingly, the inventors have found that the histamine releasing factor (HRF) was increased in relapse (FIG. 4A). Since this factor is known to enhance the production of IL13 by basophil cells (21), it may contribute to development of Th2 cells in MCNS.

The inventors' results, showing an upregulation of many genes closely involved in T-cell response, support previous studies suggesting that MCNS is a T cell-mediated disease (22, 23). Furthermore, they identify, for the first time, important signaling pathways shaping T-cell response during MCNS relapse. It is likely that the genes isolated in this library will contribute effectively to knowledge of the pathophysiology of MCNS.

Material and Methods for Examples 3 to 8

1) Patients: A cohort of patients in a previous study (5), analyzing the activity of NFK-B in the regulation of cytokine expression during the relapse and remission phases of steroid sensitive MCNS, formed the basis of this study. In children, the criteria of the international study of kidney disease were used for diagnostic and management of MCNS (7). In adults, the diagnostic of MCNS or Membranous Nephropathy (MN) was confirmed by renal biopsy before inclusion. Blood sampling of patients with relapse was performed before any treatment except when indicated. Informed consent was obtained from the parents and whenever possible from the pediatric patients, as well as from normal volunteers. Controls consisted of normal children studied while undergoing routine analysis, and normal adult volunteers.

All patients (children's and adults) have had a proteinuria above 3 g/24 h, and a severe hypoalbuminemia.

Relapse was defined by sudden onset of the nephrotic syndrome (proteinuria with at least 3+ protein by urine dipstick, for three consecutive days), in a patient previously free of proteinuria, regardless of therapy. In all cases, the diagnostic of nephrotic relapse was established at the time of blood sampling. Remission was defined by the disappearance of nephrotic syndrome with a proteinuria below 0.2 g/24 h.

Informed consent was obtained from the parents and whenever possible from the children patients, as well as from MN patients and normal volunteers.

2) Purification of PBMC and T cell subsets. Peripheral blood mononuclear cells (PBMC) were purified through a Ficoll/Hypaque gradient density. The CD8⁺ T cells and monocytes enriched populations were purified from PBMC by positive selection, using CD8 and CD14 microbeads, respectively. Then, CD4⁺ T cells enriched population was collected by immunomagnetic negative selection using a cocktail of hapten-conjugated CD8, CD11b, CD16, CD19, CD36, and CD56 antibodies and MACS Microbeads coupled to an anti-hapten monoclonal (Miltenyi Biotech, Inc). The purity of preparation was of 90-95%, as assessed by flow cytometric analysis.

3) Immunocytochemistry. PBMC, T cell subsets, and monocytes were spread at 10⁵ cells/slide, fixed and permeabilized by methanol at −20° C., then processed for immunoreactivity. Cells were incubated with the blocking solution (10% sheep normal serum, 1% BSA) for 40 min, washed twice with PBS, then allowed to react with c-Maf antibody (10 μg/ml in 5% sheep normal serum, 1% BSA, 0,1% Triton 100) for 2 hours at room temperature. Following 3 washs, cells were incubated with anti rabbit antibody cy3-labelled (1/1000 in blocking solution) for 30 min. Slides were mounted in a Vectashield DAPI (Vector laboratories, Incn, Burlingame, USA), and analyzed on a Axioplan Zeis microscope equipped for epifluorescence coupled with a camera (Hamamatsu 3 CCD).

4) Reverse transcription-PCR (RT-PCR): Total RNA was isolated from PBMCs, T cell subsets and monocytes, using Rneasy kit (Qiagen SA). The sequence of primers, and PCR characteristics are indicated in table 3. In order to determine the relative expression of each cmaf mRNA species in MCNS, the inventors selected two sets of primers. Given that both forms of cmaf mRNA were identical in 5′non-coding and in coding sequence until to stop codon of short form (position 1926), the only possibility to specifically analyze the short form is to select a set of primers in 5′untranslated sequence, which are designed in table 3. For the long form cmaf mRNA, the sens primer was common for both forms but the antisens primer was located downstream of short form coding sequence, so that only the long form could be amplified. Semiquantitative RT-PCRs were performed as previously described (5) with the following conditions: denaturation 94° C., 1 min; annealing 60° C., 30 sec and extension 68° C., 2 min. The expression level of GATA-3 and Maf B was analyzed in parallel, using the primers listed in table 3, under similar PCR conditions. Southern blots of amplified products were detected with specific internal oligonucleotides and quantified using the ImageQuant v1.11 analysis software. PCR reactions were normalized for GAPDH expression, in order to control the variations in the RT reaction.

5) Quantification of IL4 mRNA by quantitative RT-PCR. Quantitative PCR was performed using the Light Cycler (Roche Molecular Biochemical). The samples (2 μl of the RT reaction, corresponding to 20 ng of total RNA) were amplified in a 20 μl reaction mixture containing 0.5 mM each primer, and 1×mix LightCycler-DNA master SYBR Green buffer (Roche Molecular Biochemical). Carry-over was prevented by using dUTP instead of dTTP, and heat-labile Uracil DNA glyco-sylase (UDG). A standard curve was made using dilution of the RNA prepared from pUc9-IL4 plasmid. The PCR reaction was started by a denaturation at 95° C. for 2 min followed by 40 three step cycles (95° C.: 1 sec, 60° C.: 10 sec, and 72° C.: 24 sec). The relative value for each sample was calculated using the LightCycler analysis software. 6) Electromobility shift assays (EMSA), immunoprecipitations and Western blottings: Cytosolic and nuclear protein preparations as well as EMSA were performed as previously described (34). The MARE oligonudeotde (5′-GGMTTGCTGACTCAGCATTACT-3′ [SEQ ID NO:5]) containing the c-Maf recognition sequence (in bold characters) was synthesized (Genset, France). Specificity of the binding was tested by the addition of 50-fold molar excess unlabelled c-Maf and by supershift assays. The subunit composition of DNA-protein complexes containing c-Maf was determined by preincubation of nuclear extracts with 1 μg of polyclonal antibodies raised against c-Maf (sc-7866), c-Fos (sc-7202), and c-Jun (sc-1694), (Santa Cruz, Biotechnology), before the addition of the probe. Gels were dried and revealed after overnight exposition on a Phosphoimager (Storm 840, Molecular dynamics SA). Band shifts were quantified using the ImageQuant V1.11 analysis software. Immunoprecipitations and Western lotting were performed as previously described (34).

7) Incubation of PBMCs with the proteasome inhibitor MG 132. PBMCs were suspended in complete RPMI 1640 medium supplemented with 10% heat-inactivated fetal calf serum, 50 μg/ml penicillin and 100 μg/ml streptomycin in a humidified incubator containing 5% CO₂, at a concentration of 2×10⁶ cells/ml. Cells were subdivided in two equal fractions and incubated overnight, in the absence (control) or in the presence of 10 μM of proteasome inhibitor MG132 (carbobenzoxyl-leucinyl-leucinyl-leucinal-H, Z-LLL) (Calbiochem) at 37° C. Following incubation, cells were pelleted by centrifugation at 1200 g for 10 min and washed three times with ice cold phosphate buffered saline (PBS) and the protein extracts were prepared.

8) Data analysis. Results were analyzed using the ANOVA program. Statistical significance was determined by using the non-parametric Mann-Whitney test or the wilcoxon's test for apparied data.

### Example 3

### Isolation of c-maf by Subtractive and Differential Cloning

The inventors have generated a cDNA library enriched for transcripts induced in MCNS relapse through two major steps. First, the inventors have performed a subtractive cloning of cDNA synthesized from PBMC isolated in relapse and in remission from the same patient with proven biopsy MCNS. Second, the inventors have selected for analysis subtracted cDNAs that react with relapse MCNS but not with remission or unrelated nephrotic syndrome (membranous nephropathy) probe. The sequencing of both extremities of subtracted cDNA clones have revealed that 25% of selected transcripts, including c-maf, are encoding parts of genes involved in T cell signaling patways.

### Example 4

### Specific Induction of c-maf in MCNS

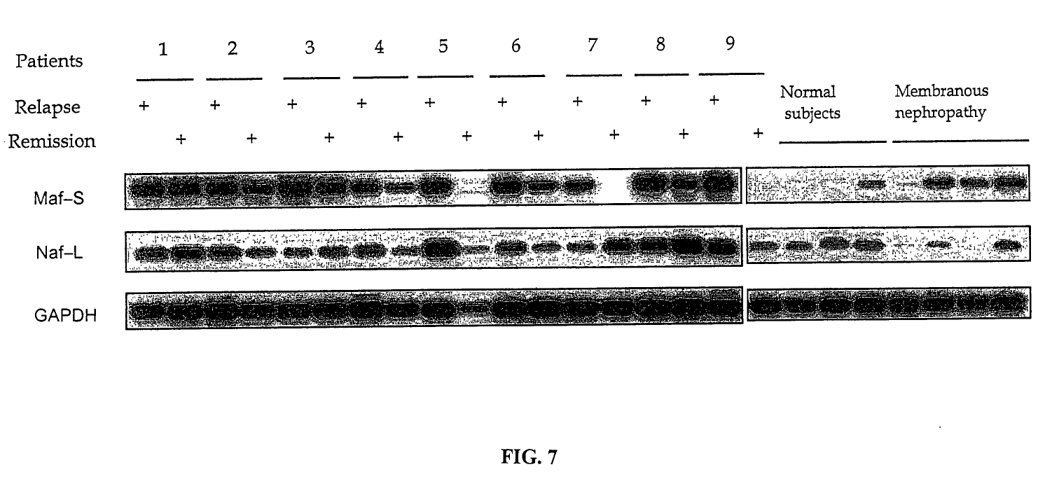

To confirm this result, the inventors analyzed the expression of c-maf in PBMC of seven patients in relapse and remission phases (FIG. 7). A strong induction of c-maf mRNA was detected in all patients with MCNS, compared to five normal controls. This induction was not an effect of nephrotic syndrome itself, seeing that membranous nephropathy patients who suffered of a similar range of proteinuria exhibited a c-maf mRNA level undistinguable from controls.

Although each patient exhibited a proper expression profile, the c-maf mRNA level was constantly increased in relapse. In remission, two types of expression profiles were detected. In patients No. 2, 3, and 4, the expression level of c-maf was low. In patients No. 1, 5, 6, and 7, the expression level between relapse and remission phases was not significantly different. Indeed, these expression profiles corresponded to distinct clinical patterns (see below).

### Example 5

### The Burst of c-maf Induction was Restricted to CD4 T Cells

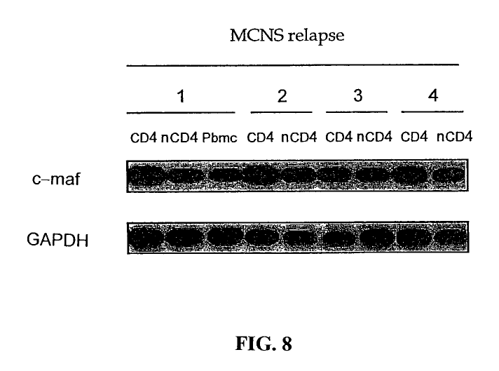

To determine whether the expression of c-Maf was associated with a particular cell subset, the inventors purified by immunomagnetic selection the CD4⁺, and CD8⁺T cells as well as monocyte fractions from PBMC of patients with nephrotic relapse. The highest level of c-maf mRNA was observed in CD4⁺T cell subset (FIG. 8). CD8⁺T cell subset exhibited a more discrete level whereas in monocyte, c-maf mRNA was barely detected.

### Example 6

### The Induction of c-maf in MCNS Relapse was not Associated with an Uprequlation of GA TA-3 or maf B

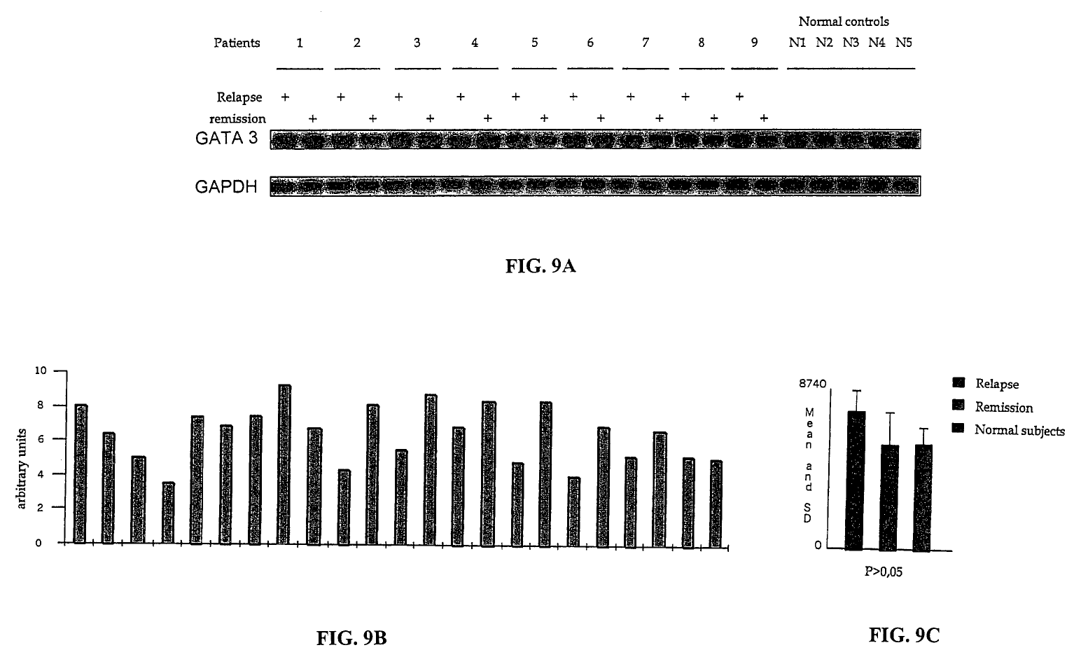

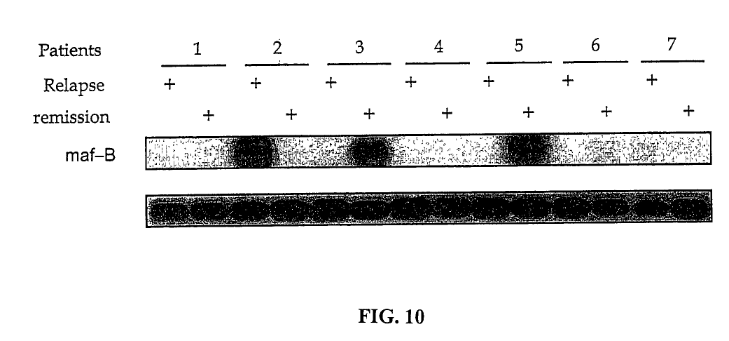

The inventors previously have shown that the IL12Rβ2, signaling component of IL12R was downregulated during the relapse, suggesting that the MCNS is associated with a Th2 T cell expression disease. It is believed that the transcription factor GATA-3 is the master factor that drives the naive CD4 T cells along the Th2 pathway. In the same line of argument, the inventors looked if the relapses were associated with the expression of maf B, a member of maf family, which is specifically induced during monocytic and macrophage differentiation. Thus, the inventors sought to determine if the induction of c-maf during the relapse was associated with an upregulation of GATA-3 and maf B, of which the expression was analyzed in parallel. The inventors showed that the Gata3 transcript does not exhibit significant difference of expression between relapse and remission phases, as compared to normal controls (FIG. 9A, FIG. 9B and FIG. 9C). Moreover, the expression of Maf B transcript was not correlated with clinical course of the disease, given the lack of detectability in most patients (FIG. 10).

### EXAMPLE 7

### C-maf Protein Induction was Correlated with c-maf mRNA Levels

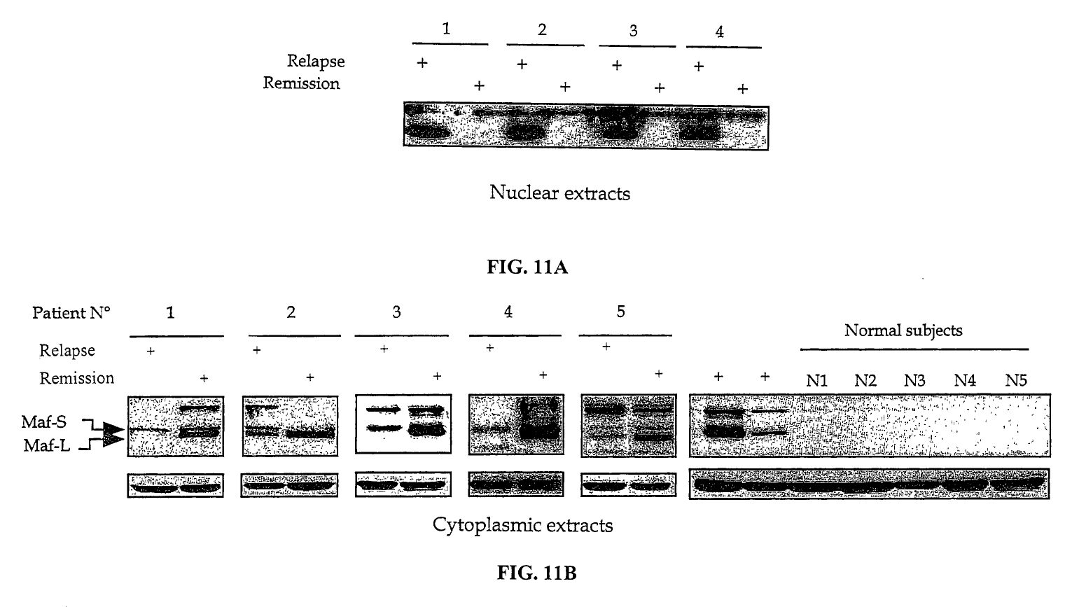

The c-maf gene encodes two predicted form proteins of 373 and 403 aa, generated by alternative splicing. To assess whether the induction of c-maf mRNA was correlated with the expression of c-maf protein, the inventors performed immunoblotting with cytoplasm extracts from PBMC or T cell fractions, using an antibody reacting with the transactivation domain of c-maf. This antiserum detected two bands of 42-45 Kda that are in good agreement with the predicted molecular weight (FIG. 11B). They correspond to short and long form c-maf proteins expressed in relapse with a variable level that is consistently higher in the same patient during remission. On the other hand, c-maf was barely or not detected in normal subjects as well as in nephrotic membranous nephropathy patients. The additional slow migrating band of 80 Kda detected in most patients is likely non specific as confirmed by immunoprecipitation experiments (data not shown). In parallel the inventors analyzed the expression of c-maf in cell compartments during the disease. As depicted in FIG. 11A, the inventors showed that c-maf was expressed in nuclear compartment only during the relapse phase.

### Example 8

### Activation of c-maf in MCNS Relapse but not in Remission

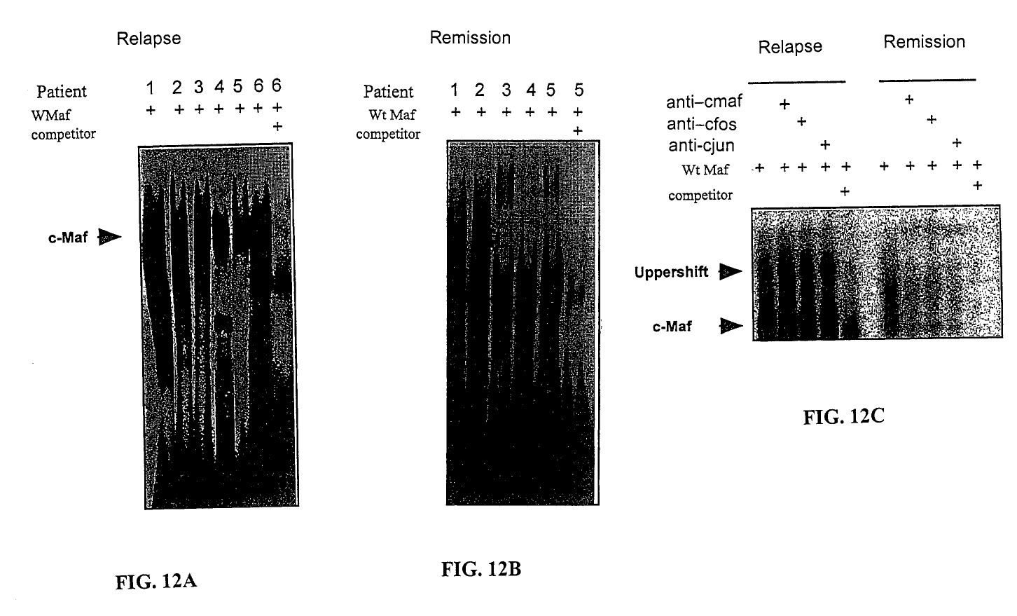

The induction of c-Maf protein in MCNS relapse, and its persistence in remission raised the question of the subcellular localization of c-maf in both situations. Indeed, it is important to determine whether the transactivator function of c-maf operates in relapse and in remission. For this, the inventors analyzed c-Maf binding activity, and the expression of c-maf in cell compartments during the disease. EMSA experiments were performed with a consensus Maf responsive element (MARE) probe. Nuclear extracts from relapse exhibit a striking Maf binding activity in contrast to remission (FIG. 12A, FIG. 12B and FIG. 12C). The significance of the lower migrating complexes with the MARE probe, observed in nuclear extracts from remission, likely correspond to small Maf homodimers which act as repressor of c-Maf activity (27). The specificity of the binding was attested by competition with unlabelled oligonucleotide, and by antibody uppershift (FIG. 12C). The subunit composition of MARE-complexes was analyzed by preincubation of nuclear extracts with antibodies raised against c-Maf, c-Fos, and c-Jun, respectively, before the addition of the MARE probe. The complexes were supershifted with both c-Maf, c-Fos, and c-Jun antibodies (FIG. 12C). In contrast, the MARE binding activity was not detected and no supershift could be identified with nuclear extracts from patients in remission (FIG. 12C).

The lack of DNA binding activity dependent of c-maf may be explained by the exclusion of c-maf from the nuclear compartment, or by the inhibition of its binding to target genes upon the nuclear intrusion of small mafs, or its association with a putative inhibitor. To differentiate between these possibilities, the inventors analyzed the subcellular localization of c-maf in relapse and in remission phases. In the relapsing phase before the initiation of steroid therapy, c-maf was detected in nuclei (FIG. 11A) as well as in cytoplasm of positive cells. For patients who elicit a first relapse, the percentage of positive cells among PBMC was near of 10%. For most of patients studied, almost 50% of PBMC and 100% of CD4⁺T cell were positive for c-maf. The reasons for this discrepancy are unclear. In the remission phase, cmaf was detected exclusively in cytoplasm of CD4⁺T cell. These results suggest that the lack of DNA binding activity in remission was correlated with the exclusion of c-maf from the nuclei. On the other hand, c-maf was not detected in PBMC of normal subjects (FIG. 12A, FIG. 12B and FIG. 12C), thus confirming the data of western blotting.

TABLE 1

Sets of primers used in semiquantitative RT-PCR.
The oligonucleotides are selected from sequences of which
the accession numbers are indicated.
The expected size of each amplified sequence,
its Tm annealing, and the number
of PCR cycles are indicated.

| | | | | | |
|---|---|---|---|---|---|
| | | | expected | | |
| | | Accession | size | Tm | PCR |
| mer | oligonucleotide | number | (bp) | annealing | cycles |

| | | | | | | |
|---|---|---|---|---|---|---|
| GAPDH | 5′-ACCACAGTCCATGCCATCAC-3′ | (SEQ ID NO:6) | NM 004048 | 374 | 58° C. | 22 |
| | 5′-TCCACCACCCTGTTGCTGTA-3′ | (SEQ ID NO:7) | | | | |
| IL12Rβ1 | 5′-AGCTTCCAGAAGGCTGTCAAGG-3′ | (SEQ ID NO:8) | X03934 | 135 | 60 | 29 |
| | 5′-GCTGCCATTCAATGCAATACGTC-3′ | (SEQ ID NO:9) | | | | |
| IL12Rβ2 | 5′-AGACACCCACTTATACACTGAGTA-3′ | (SEQ ID NO:10) | XM_010533 | 514 | 60 | 32 |
| | 5′-CTCTTCTGGTGGTGTTTGTGCTCT-3′ | (SEQ ID NO:11) | | | | |
| FYB | 5′-AAAAGACTCTCTTGGTGCCCCTTC-3′ | (SEQ ID NO:12) | AF001862 | 559 | 60 | 32 |
| | 5′-CATAGATCTCTCCATCATTGTCCGC-3′ | (SEQ ID NO:13) | | | | |

TABLE 2
Transcripts upregulated in PBMC of patients with MCNS relapse.
Ten thousands clones of the forward subtracted library were
screened with different probes as indicated under Material
and Methods. One hundred twenty seven subtracted clones were
selected for partial sequencing. The number of clones (Nb)
corresponding to a given transcript is indicated.
Transcripts were identified by comparison with sequences
present in Genbank under the accession number indicated.
The genomic localizations of the corresponding
genes are reported on the right.

| | | Accession | Genomic |
|---|---|---|---|
| cDNAs | Nb | number | localization |
| Known genes | | | |
| AP-2 beta | 1 | XM_004325 | 6p12 |
| Beta actin | 1 | X00351 | 7p15-p12 |
| Bos taurus tyrosinase-related protein | 1 | L43123 | 19p23 |
| cAMP dependent protein kinase | 2 | M34181 | 1p36.1 |
| Cathepsin S | 1 | BC002642 | 1q21 |
| c-Maf | 2 | AF055376 | 16q22-q23 |
| DNA binding protein A | 1 | M24069 | 12p13.1 |
| Deoxyguanosine kinase | 1 | U41668 | 2p24.3-p24.1 |
| Elongation factor 1-Alpha 1 | 1 | M29548 | 6q14 |
| Ferritin | 2 | M11146 | 11q13 |
| Fyn-T binding protein (FYB)120 | 4 | AF001862 | 5p13.1 |
| Grancalcin | 2 | M81637 | 2 |
| Heat shock protein 71 | 2 | Y00371 | 11q23.3-q25 |
| Heat shock protein 90 | 3 | D87666 | 19q21.2-q22 |
| HLA F | 3 | X17093 | 6p21.3 |
| Homologue to GDP-dissociation for | 2 | L07916 | 12p12.3 |
| the Rho GTP-bound protein | | | |
| Human membrane bound amino- | 1 | AF195953 | — |
| peptidase P | | | |
| IgE dependent Histamine releasing | 3 | X16064 | 13q12-q14 |
| factor | | | |
| IL1-beta | 1 | NT_019306 | 2q14 |
| IL7 receptor | 2 | M29696 | 5q13 |
| Inhibitor of p53-induced apoptosis- | 1 | U90450 | 5q31 |
| beta (IPIA-beta) Mrna | | | |
| Initiation factor 4B | 1 | X55733 | 12q13-q14 |
| Interferonα receptor type 2 | 2 | L42243 | 21q2.11 |
| Jak1 | 2 | M64174 | 1p32.3p31.3 |
| low density lipoprotein receptor | 1 | XM_006874 | 12p11-p13 |
| (LRP6) | | | |
| L13 protein | 2 | BC007345 | 16q24.3 |
| L-Plastin | 3 | L05492 | 13q14.3 |
| Macropain | 3 | D00760 | 2q33 |
| NFAT5 | 2 | AF134870 | 16q22.1 |
| NADH ubiquinone oxidoreductase | 3 | AF164796 | 14 |
| (MLRQ subunit) | | | |
| Nuclear factor related to kappa B | 1 | NM 006165 | 11q24-q25 |
| binding protein (NFRKB), mRNA. | | | |
| P38 Map kinase | 2 | L35253 | 6p21.3p21.2 |
| Promyelocytic leukemia cell mRNA | 3 | M11948 | X |
| Ras Gap related protein | 2 | U51903 | 5q |
| Rho A protein | 1 | L09159 | 3p21.3 |
| Selectin L | 2 | XM001577.1 | 19q23-q25 |
| Small inducible cytokine | 3 | NM002985 | 17q11.2-q12 |
| A5 (RANTES) (SCYA5), mRNA | | | |
| T cell receptor | 1 | M18414 | 14q11.2 |
| T cell receptor | 1 | AE000661 | 14q11 |
| T cell receptor | 1 | X15260 | — |
| Thiopurine methyl transferase | 1 | U81566 | 6p22.3 |
| TRAF6 | 1 | XM_006284 | 11pter-p15.5 |
| Genes with protein product | | | |
| of unknown function | | | |
| 1-8 d | 2 | X57351 | 11 |
| cDNA related to DC10 | 1 | AF201932 | 4 |
| cosmid Q7A10 | 5 | D42052 | 21q22.11 |
| HSPC025 | 2 | AF083243 | 22q |
| Homo sapiens hypothetical protein | 2 | AF119891 | 3 |
| PRO2706 mRNA | | | |
| Human RNA for KIAA0121 gene | 1 | D50911 | 3 |
| Human RNA for KIAA0228 gene | 1 | D86981 | 17q21-q23 |
| Human RNA for KIAA0386 gene | 1 | XM011388 | 6p22.3-p21.32 |
| Human RNA for KIAA0530 gene | 1 | AB011102.1 | 6 |
| Homo sapiens GL004 protein | 3 | AF226049 | 5 |
| (GL004), mRNA | | | |
| Human zinc finger protein GLI1 | 1 | P08151 | 12q13.2-q13.3 |
| Novel human gene mapping to | 2 | AL117237 | 1q12.1-q21.2 |
| chromosome 1 | | | |
| Unknown sequences (30) | | | |
| no data base match | 1- | | |
| | 2 | | |

TABLE 3

Sets of primers used in semiquantitative RT-PCR.
The oligonucleotides are selected from sequences of which the
accession numbers are indicated on the right.
The size of each amplified sequence, its annealing temperature,
and the number of PCR cycles are indicated.

| | | | | | |
|---|---|---|---|---|---|
| | | Accession | expected | Tm | PCR |
| mRNA | oligonucleotides | number | size (bp) | annealing | cycles |

| | | | | | | | |
|---|---|---|---|---|---|---|---|
| GAPDH | S | 5′-ACCACAGTCCATGCCATCAC-3′ | (SEQ ID NO:14) | NM004048 | 374 | 58° C. | 25 |
| | AS | 5′-TCCACCACCCTGTTGCTGTA-3′ | (SEQ ID NO:15) | | | | |
| | i | CTCAAGGGCATCCTGGGCTACACTGAGCAC | (SEQ ID NO:16) | | | | |
| C-MAF | S | 5′-TGCACTTCGACGACCGCTTCTC-3′ | (SEQ ID NO:1) | AF055376 | | 60 | 29 |
| | AS | 5′-CGCTGCTCGAGCCGTTTTCTC-3′ | (SEQ ID NO:2) | | | | |
| | | (Short form) | | | | | |
| | AS2 | 5′-GGTGGCTAGCTGGAATCGCG-3′ | (SEQ ID NO:3) | AF055377 | | | |
| | AS3 | (long form) | | | | | |
| | | 5′-TGTACAGCTCTCACACAAATTTCATTTTGT-3′ | (SEQ ID NO:4) | | | | |
| C‘maf5’UTR | U5 | TGTGGGCTTGCTAGTTCTAGAGCCATGCTCG | (SEQ ID NO:17) | AF055376 | | | |
| | U3 | CACAAGTCACACCCAGAAGGTTGATGCAGGC | (SEQ ID NO:18) | | | | |
| | U15 | CTCCCAATGCACTGAAGGCATTCCTTG | (SEQ ID NO:19) | | | | |
| | U13 | GTCCCCTTGCAAACTCTACCCCCCTTAAC | (SEQ ID NO:20) | | | | |
| ST2 | S | 5′-CGAGACCGAATACCAGGTGATCGGAG-3′ | (SEQ ID NO:21) | BC002443 | 498 | 60 | 34 |
| | AS | 5′-CTTGTCCTGGAAGAAGCGCTTGAGCG-3′ | (SEQ ID NO:22) | | | | |
| | i | GACATCAAGGAGTCCATTGAGACCATGCG | (SEQ ID NO:23) | | | | |
| GATA3 | S | 5′-CTGGAATCTCAGCCCCTTCTCCAAGACG-3′ | (SEQ ID NO:24) | BC006793 | 476 | 60 | 32 |
| | AS | 5′-GTTGCCCCACAGTTCACACACTCCCTG-3′ | (SEQ ID NO:25) | | | | |
| | | GGCTCGGGCCGGCAGGACGAGAAAGAGTGC | (SEQ ID NO:26) | | | | |
| Maf B | S | ATGGAGTATGTCAACGACTTCGACCTG | (SEQ ID NO:27) | AF134157 | 925 | 60 | 38 |
| | AS | ACGACTCACAGAAAGAACTCGGGAG | (SEQ ID NO:28) | | | | |
| | | CTGCGGGGCTTCACCAAGGACGAGGTGAT | (SEQ ID NO:29) | | | | |

### REFERENCES

1. Savin V. J. 1993. Mechanisms of proteinuria in noninflammatory glomerular diseases. Am J Kidney Dis. 21: 347.

2. Yap, H. K., W. Cheung, B. Murugasu, S. K. Sim, C. C. Seah, S. C. Jordan. 1999. Th1 and Th2 cytokine mRNA profiles in childhood nephrotic syndrome: evidence for increased IL-13 mRNA expression in relapse. J Am Soc Nephrol 10: 529.

3. Bustos, C., E. Gonzalez, R. Muley, J. L. Alonso, J. Egido. 1994. Increase of tumour necrosis factor alpha synthesis and gene expression in peripheral blood mononuclear cells of children with idiopathic nephrotic syndrome. Eur J Clin Invest 24: 799.

4. Garin, E. H., D. K. Blanchard, K. Matsushima, J. Y. Djeu. 1994. IL-8 production by peripheral blood mononuclear cells in nephrotic patients. Kidney Int 45: 1311.

5. Sahali D., Pawlak A, Ie Gouvello S, Lang P, Valanciuté A, Remy P, Loirat C, Niaudet P, Bensman A, and Guellaen G. 2001. Transcriptional and Post-transcriptional Alterations of I [symbol] B [symbol] in Active Minimal Change Nephrotic Syndrome. J. Am Soc. Nephrol. (2001, 12(8):1648-58.

6. The primary nephrotic syndrome in children. 1981. Identification of patients with minimal change nephrotic syndrome from initial response to prednisone. A report of the International Study of Kidney Disease in Children. J Pediatr. 98: 561.

7. Diatchenko L, Lau Y F, Campbell A P, Chenchik A, Moqadam F, Huang B, Lukyanov S, Lukyanov K, Gurskaya N, Sverdlov E D, and Siebert P D. 1996. Suppression subtractive hybridization: a method for generating differentially regulated or tissue-specific cDNA probes and libraries. Proc Natl Acad Sci U S A. 93: 6025.

8. Maniatis T, Fritsch E F, and Sambrok J. 1989. Molecular cloning: A laboratory manual. Cold Spring Harbor Laboratory Press, Cold Spring Harbor, N.Y.

9. Bernard K, Auphan N, Granjeaud S, Victorero G, Schmitt-Verhulst A M, Jordan B R, and Nguyen C. 1996. Multiplex messenger assay: simultaneous, quantitative measurement of expression of many genes in the context of T cell activation. Nucleic Acids Res. 24: 1435.

10. Pawlak A, Toussaint C, Levy I, Bulle F, Poyard M, Barouki R, and Guellaen G. 1995. Characterization of a large population of mRNAs from human testis. Genomics. 2:151.

11. Penninger J M, and Crabtree G R. 1999. The actin cytoskeleton and lymphocyte activation. Cell. 96: 9.

12. Henning, S W, Meuer S C, and Samstag Y. 1994. Serine phosphorylation of a 67-kDa protein in human T lymphocytes represents an accessory receptor-mediated signaling event. J Immunol. 152: 4808.

13. Jia J, Han Q, Borregaard N, Lollike K, and Cygler M. 2000. Crystal structure of human grancalcin, a member of the penta-EF-hand protein family. J Mol Biol. 300: 1271.

14. Maesaki R, Ihara K, Shimizu T, Kuroda S, Kaibuchi K, and Hakoshima T. 1999. The structural basis of Rho effector recognition revealed by the crystal structure of human RhoA complexed with the effector domain of PKN/PRK1. Mol Cell. 4: 793.

15. Rodig S J, Meraz M A, White J M, Lampe P A, Riley J K, Arthur C D, King K L, Sheehan K C, Yin L, Pennica D, Johnson E M Jr, and Schreiber RD. 1998. Disruption of the Jak1 gene demonstrates obligatory and nonredundant roles of the Jak1 in cytokine-induced biologic responses. Cell. 93: 373.

16. Porter B O, Scibelli P, Malek T R. 2001. Control of T cell development in vivo by subdomains within the IL-7 receptor-chain cytoplasmic tail. J Immunol. 2001, 166: 262.

17. Liu J, Kang H, Raab M, Da Silva A J, Kraeft S K, and Rudd C E. 1998. FYB (FYN binding protein) serves as a binding partner for lymphoid protein and FYN kinase substrate SKAP55 and a SKAP55-related protein in T cells. Proc Natl Acad Sci U S A. 95: 8779.

18. Trama J, Lu Q, Hawley R G, Ho S N. 2000. The NFAT-related protein NFATL1 (TonEBP/NFAT5) is induced upon T cell activation in a calcineurin-dependent manner. J Immunol. 165: 4884.

19. Szabo S J, Dighe A S, Gubler U, Murphy K M. 1997. Regulation of the interleukin (IL)-12R□2 subunit expression in developing T helper1 (Th1) and Th2 cells. J Exp. Med. 185:817.

20. Rogge L, Maino L B, Biffi M, Passini N, Presky D H, Gubler U, Sinigaglia F. 1997. Selective expression of an interleukin-12 receptor component by human T helper 1 cells. J Exp. Med. 185:825.

21. Schroeder J T, Lichtenstein L M, MacDonald S M. 1997. Recombinant histamine-releasing factor enhances IgE-dependent IL4 and IL13 secretion by human basophils. J Immunol. 159: 447.

22. Shalhoub R J. 1974. Pathogenesis of lipoid nephrosis: a disorder of T-cell function. Lancet. 2: 556.

23. Daniel V, Trautmann Y, Konrad M, Nayir A, Scharer K. 1997. T-lymphocyte populations, cytokines and other growth factors in serum and urine of children with idiopathic nephrotic syndrome. Clin Nephrol. 47: 289.

What is claimed is:

1. A method for diagnosing the occurrence of Minimal Change Nephrotic Syndrome (MCNS) in a human, wherein said method comprises the steps of:
a) collecting a biological sample from said patient;
b) quantifying the expression level of the c-Maf gene in the biological sample obtained at step a); and
c) comparing the expression level of the c-Maf gene quantified at step b) with the expected expression level of said gene in patients not affected with MCNS.

2. The method of claim 1, wherein step b) consists of quantifying the mRNA transcribed from the c-Maf gene in said biological sample.

3. The method of claim 2, wherein the step of quantifying the mRNA transcribed from the c-Maf gene is performed by a RT-PCR reaction using a pair of primers hybridizing specifically with the c-Maf cDNA.

4. The method of claim 2, wherein the biological sample consists of Peripheral Blood Mononuclear Cells (PBMC).

5. The method of claim 1, wherein step b) consists of quantifying the c-MAF protein contained in said biological sample.

6. The method of claim 5, wherein the step of quantifying the c-MAF protein is performed by incubating at least one antibody which recognizes specifically said protein with said biological sample.

7. The method of claim 5, wherein the step of quantifying the c-MAF protein is performed by immunofluorescence using at least one antibody which recognizes specifically said protein, and wherein said antibody is fluorescently labeled, either directly or indirectly.

8. The method of claim 5, wherein the step of quantifying the c-MAF protein is performed by incubating at least one antibody which recognizes specifically said protein with a protein extract obtained from said biological sample and detecting the complexes formed between said antibody and the c-MAF protein contained in the biological sample.

9. The method of claim 5, wherein the step of quantifying the c-MAF protein is performed by incubating at least one antibody which recognizes specifically said protein with cells which are contained in said biological sample and detecting the localization of the complexes formed between said antibody and the c-MAF protein within said cells.

10. The method of claim 9, wherein the cells contained in said biological sample consists of PBMCs.

11. The method of claim 5, wherein the step of quantifying the c-MAF protein is performed by incubating said biological sample consisting of a protein extract with a consensus Maf responsive element (MARE) probe and detecting the complexes formed between the MARE probe and the c-MAF proteins contained in the biological sample.

12. The method of claim 11, wherein the MARE probe consists of the nucleotide sequence SEQ ID NO:5.

13. A method for distinguishing between a MCNS remission phase and a MCNS relapse phase in a human patient affected with Minimal Change Nephrotic Syndrome, wherein said method comprises the steps of:
a) collecting a biological sample from said patient; and
b) quantifying the short form of the c-MAF protein respectively in (i) the cell nucleus and (ii) in the whole-cell or the cell cytoplasm from the cells contained in said biological sample.

14. The method of claim 13, wherein step b) of quantifying the short form of the c-MAF protein is performed by incubating at least one antibody which recognizes specifically the c-MAF protein with the cells contained in the biological sample and detecting the complexes formed between said antibody and the short form of the c-MAF protein respectively localized within the nucleus and within the cytoplasm of said cells.

15. The method of claim 14, wherein the cells contained in the biological sample consist of PBMCs.

16. The method of claim 13, wherein step b) of quantifying the short form of the c-MAF protein is performed by incubating at least one antibody which recognizes specifically the c-MAF protein with respectively (i) a nuclear extract and (ii) a whole cell extract or a cytoplasm extract obtained from the cells contained in the biological sample and detecting the complexes formed between said antibody and the short form of the c-MAF protein contained in said extracts.

17. The method of claim 16, wherein the cells contained in the biological samples consists of PBMCs.

18. The method of claim 13, wherein step b) of quantifying the c-MAF protein is performed by incubating respectively (i) nuclear extracts and (ii) whole cell extracts or cytoplasm extracts obtained form the cells contained in the biological sample with a consensus Maf responsive element (MARE) probe and detecting the complexes formed between the MARE probe and the c-MAF proteins contained in said extracts.

19. The method of claim 18, wherein the MARE probe consists of the nucleotide sequence SEQ ID NO:5.

20. A kit for diagnosing the occurrence of Minimal Change Nephrotic Syndrome (MCNS) or for diagnosing the occurrence of a MCNS remission phase or a MCNS relapse phase in a human, wherein said kit comprises an antibody which recognizes specifically the c-MAF protein.

21. The kit of claim 20, which comprises an antibody which recognizes specifically the c-MAF protein.

22. The kit of claim 20, wherein said antibody is labeled with a detectable molecule.

23. The kit of claim 20, wherein said antibody is labeled with a fluorescent molecule.

24. A kit for diagnosing the occurrence of Minimal Change Nephrotic Syndrome (MCNS) or for diagnosing the occurrence of a MCNS remission phase or a MCNS relapse phase in a human, wherein said kit comprises a a consensus Maf responsive element (MARE) probe.

25. The kit of claim 23, wherein the MARE probe consists of the nucleotide sequence SEQ ID NO:5.

26. A kit for diagnosing the occurrence of Minimal Change Nephrotic Syndrome (MCNS) in a human, wherein said kit comprises a pair of primers hybridizing specifically with the c-Maf cDNA.
